(19) United States
(12) Patent Application Publication
(10) Pub. No.: US 2008/0008670 A1
(43) Pub. Date: Jan. 10, 2008

(54) Process for colouring dark skin

(75) Inventors: Pascal Arnaud, L'hay Les Roses (FR); Charlotte Feltin, Paris (FR); Eve Bazelaire, Joinville-Le-Pont (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 11/806,049

(22) Filed: May 29, 2007

Related U.S. Application Data
(60) Provisional application No. 60/834,161, filed on Jul. 31, 2006.

(30) Foreign Application Priority Data
May 29, 2006 (FR) 06 51940
Jan. 12, 2007 (FR) 07 52649

(51) Int. Cl. A61K 8/06; A61Q 1/02; A61Q 1/12

(52) U.S. Cl. 424/63

(57) ABSTRACT

The present invention relates to a process for making up dark skin, comprising the application to the said skin of a composition comprising, in a physiologically acceptable medium, at least one direct colouring system in dissolved form, the said composition also having a viscosity of greater than 100 centipoises and a covering power, defined by the Contrast Ratio, of less than 80.

This non provisional application claims the benefit of French Application No. 06 51910 filed on May 29, 2006, French Application n° 07 52649 of Jan. 12, 2007 and U.S. Provisional Application No. 60/834,161 filed on Jul. 31, 2006.

The present invention is directed towards proposing cosmetic compositions to be applied to the skin, and more particularly compositions intended for dark skin, including skin of black and mixed-race type.

In general, cosmetic products and in particular those intended for makeup are aimed at affording an aesthetic effect, frequently a colour effect, on the makeup support, or even a covering effect with regard to cutaneous defects of the surface to be made up.

To afford such effects, cosmetic compositions generally combine mineral or organic fillers and colouring agents.

Fillers are conventionally used to modify the rheology, to adjust the texture of the composition or to reduce its gloss, and/or as matting agents intended especially for affording an advantageous covering effect to hide skin imperfections. These fillers are thus conventionally chosen as a function of their colouring property, their covering power or their geometry, which allow light to be scattered efficiently. They are generally in the form of mineral or synthetic, colourless or white particles of any form, which are solid in the medium of the composition irrespective of the temperature at which the composition is manufactured. They are more particularly silica, mica, talc and kaolin. These fillers are conventionally used in a proportion of from 1% to 80% and especially from 1% to 50% by weight relative to the total weight of the cosmetic composition.

As regards colouring agents, including pigments, they are more particularly used as opacifier and/or colouring agent and are generally present in a concentration that is sufficient to afford the desired colour.

The term “pigments” should be understood as meaning mineral or organic, white or coloured particles, which are insoluble in the hydrophilic or lipophilic liquid phase and which are intended to colour and/or opacify the composition, the skin or mucous membranes.

Iron oxide and titanium oxide pigments have been known for a long time for their transient surface covering and colouring properties on the skin. They are generally used in makeup products such as foundations, mascaras, eyeliners and lipsticks. Their mean size may vary in general between 0.2 and 50 μm as a function of the desired colouring effects and of the intended use. Examples that may be mentioned include the makeup compositions containing them as described in patent applications WO 94/15580, WO 96/36309, WO 96/33690, WO 96/36323 and FR-A-2 754 708.

However, these conventional cosmetic compositions are not entirely satisfactory when they are applied to dark skin. Thus, their high content of filler, such as talc and kaolin, and of pigments, for example such as TiO₂, has the effect of producing on this type of makeup support a grayish or even ashen effect, which for obvious aesthetic reasons is particularly undesirable.

Now, it is clear that reducing the amount of filler and/or pigment, which might be beneficial in terms of makeup radiance, would otherwise risk being detrimental to the covering power additionally sought in order, for example, to mask dyschromia.

Furthermore, the colour effect afforded by this type of conventional composition is liable to change over time and especially to become non-uniform in response to the secretion of sebum and/or sweat.

Consequently, there is at the present time a need for a makeup composition more particularly intended for dark skin, which makes it possible to give this type of skin good colour uniformity and satisfactory staying power over time.

Document EP 1 433 460 proposes a makeup composition more particularly intended for dark skin, which combines colouring agents with reflective particles, but for the purpose of giving this type of skin a lightening effect, which is thus different from the effect sought according to the invention.

More specifically, according to one of its aspects, the present invention relates to a process for making up dark skin, comprising the application to the said skin of a composition comprising, in a physiologically acceptable medium, at least one direct colouring system in dissolved form, the said composition having a viscosity of greater than 100 centipoises and a covering power, defined by the Contrast ratio, of less than 80.

According to the invention, the term “process for making up” do not encompass-process intended to provide a lightening effect.

According to another of its aspects, the present invention is also directed towards a process for making up dark skin, comprising the application to the said skin of a composition comprising, in a physiologically acceptable medium, at least one direct colouring agent in dissolved form, the said composition having a covering power defined by the Contrast Ratio, of less than 80, and a pH of greater than 5.

For the purposes of the present invention, the term “dark skin” means skin whose mean lightness L*, measured on the forehead, the cheekbones and the chin in the CIE 1976 colorimetric space, is less than 55. The saturation C* may be, for example, between 10 and 30 and especially between 12 and 28. The hue angle values h in degrees may be, for example, between about 38° and about 54°. The lightness values L* may be less than or equal to 50, or even 45 or 40 for darker skin type, while at the same time remaining, for the majority of skin types, greater than 30.

Dark skin types are encountered, for example, among the African, Afro-American, Hispano-American, Indian and Maghrebian populations.

These skin types may also be classified on the basis of their reactivity to the effects of solar radiation according to the scale proposed by Fitzpatrick.

According to this scale, the various existing ethnic skin types may be distinguished according to the following types:

| Type | Skin reactivity | Origin |
|---|---|---|
| I | Always burns; never tans | Celtic |
| II | Always burns, tans very little | Germanic |
| III | Burns moderately, tans gradually | European |
| IV | Burns lightly, tans very easily | Mediterranean |
| V | Rarely burns, tans deeply | Middle Eastern - |
| | | South American |
| VI | Never burns, highly pigmented | African |

The dark skin types that are more particularly the subject of the present invention belong to types IV to VI.

According to one advantageous embodiment, the compositions under consideration according to the invention afford a negative variation in lightness ΔL ranging from −20 to −1 and a variation in saturation ΔC* ranging from 0.5 to 20 when they are applied to area B11+ of a contrast card as defined in document EP 1 433 462.

This card has 3 times five areas respectively having as calorimetric coordinates, to within 15%, or even 12.5%, better still to within 10%, or even 7.5%, or even to within 5%, for L* and h, and to within 25%, better still 20%, even better still 15%, or even 10%, or even to within 5%, for C*,
first zone (B11+): L*=36.7 C*=19.81 h=47.34°
second zone (B11): L*=38.43 C*=21.76 h=46.51°
third zone (B12): L*=35.66 C*=19.78 h=46.32°
fourth zone (B12+): L*=32.98 C*=17.29 h=44.64°
fifth zone (XXX): L*=29.63 C*=15.06 h=40.340
first zone (C9): L*=45.04 C*=25.18 h=53.27°
second zone (B12): L*=35.66 C*=19.78 h=46.32°
third zone (C11): L*=38.73 C*=21.94 h=50.18°
fourth zone (C10): L*=42.19 C*=24.18 h=51.940
fifth zone (C8): L*=48.06 C*=25.97 h=53.09°
first zone (D6): L*=54.08 C*=26.70 h=57.35°
second zone (C11): L*=38.73 C*=21.94 h=50.18°
third zone (D8): L*=47.94 C*=26.18 h=56.820
fourth zone (D7): L*=51.79 C*=27.21 h=57.090
fifth zone (D5): L*=57.61 C*=26.22 h=55.09°

The measurements of the trichromatic coordinates of the composition, L*, C* and h, are performed with a Minolta® brand spectrocolorimeter, of reference CM 3700d, in reflection mode, specular inclusive, small aperture SAV 5 mm.

A film of composition is applied using a Brave Instruments thermostatically regulated automatic film applicator and a Brave Instruments calibrated spreader, film thickness: 20 μm, on a transparent Impega photocopying film. The film is left to dry for 10 minutes at 37° C. in an oven, and this film is then placed on the contrast card. The calorimetric difference between the contrast card through the transparent film and the contrast card through the film thus covered with composition is measured.
Δ=(contrast card+film of cream on the transparent film)−(contrast card with the transparent film).

Covering Power

Advantageously, the cosmetic compositions according to the invention are characterized by a covering power, defined by the Contrast Ratio, of less than 80 and in particular less than or equal to 50.

This covering power may be measured by the following method.

The measurement is performed on a film of composition spread onto an Erichsen type 24/5 contrast card, having a black background and a white background.

In the case of a stick, the composition is the blended beforehand so as to obtain a viscous paste.

In the case of a powder, 50 parts by weight of the powder are blended with 50 parts by weight of dimethicone (DC 200 Fluid 5 cSt from Dow Corning) so as to obtain a viscous paste.

The film is spread to a thickness of 50 μm and the trichromatic coordinates (X, Y and Z) are measured using a CM-2002 or CR-3700 colorimeter.

Similar spreadings are made on two other contrast cards and three measurements are taken on each card. The mean corresponding to these nine measurements is then calculated.

The covering power is equal to 100×Yb/Yw where Yb is the mean value of Y on the black background and Yw is the mean value of Y on the white background.

The higher the value of the ratio, the greater the covering power.

Direct Colouring System

The term “direct colouring system” denotes a colouring agent or mixture of colouring agents which, as soon as it is placed in contact with the makeup support, generally the skin, instantaneously affords an associated coloration effect.

Compounds that exert a self-tanning action on human skin, such as derivatives containing a ketone group, for instance hydroxymethyl ketones and in particular dihydroxyacetone (DHA) and also methylglyoxane, are thus excluded from this definition. Specifically, as opposed to the direct colouring system according to the invention, these compounds produce a non-immediate colour effect.

The inventors have thus found, unexpectedly, that the use of a direct colouring system as defined above in a soluble form makes it possible to obtain a unifying and radiant result in makeup terms without having any dull or ashen nature.

Moreover, the use of such a system makes it possible to substantially increase the staying power of the makeup over time, especially in terms of its resistance to sweat and/or sebum.

Thus, the cosmetic compositions under consideration according to the invention can give dark skin a coloration with very good staying power that is advantageously distinguished from more superficial colorations that are liable to change, afforded by conventional makeup compositions, and which are generally formulated essentially using pigments in dispersion.

The colouring agent(s) forming the direct colouring system under consideration in the context of the present invention may be present in a content ranging from 0.001% to 5%, especially from 0.01% to 3% and more particularly from 0.025% to 1% by weight relative to the total weight of the composition.

The direct colouring system may represent at least 30% by weight, in particular at least 50% by weight, especially at least 65% by weight, more particularly at least 70% by weight, or even at least 75% by weight and more particularly at least 90% by weight of all of the colouring agents present in the composition.

According to one particular embodiment, this direct colouring system includes all of the colouring agents of the said composition.

As stated previously, the direct colouring system is in dissolved form in the composition, as opposed to conventional formulations in which the colouring agents are essentially formulated in a dispersed form.

The colouring agents forming the direct colouring system may thus be liposoluble or water-soluble. They are advantageously water-soluble.

The colouring agents of the direct colouring system may be synthetic or natural.

They may be organic or mineral dyes.

The natural or synthetic liposoluble dyes are, for example, DC Red 17, DC Red 21, DC Red 27, DC Green 6, DC Yellow 11, DC Violet 2, DC Orange 5, Sudan red, carotenes (β-carotene or lycopene), xanthophylls (capsanthin, capsorubin or lutein), palm oil, Sudan brown, quinoline yellow, annatto and curcumin.

The natural or synthetic water-soluble dyes are, for example, FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beetroot), carmine, copper-containing chlorophylline, methylene blue, anthocyanins (enocyanin, black carrot, hibiscus or elder), caramel and riboflavin.

The dyes may also be chosen from juglone, lawsone, extracts of fermented soya, of algae, of fungi or of microorganisms, flavylium salts not substituted in position 3, for instance those described in patent EP 1 172 091, extracts of Gesneria fulgens, Blechum procerum or Saxifraga and pigments that may be obtained by extraction with an organic or aqueous-organic solvent of a culture medium of micromycetes of the Monascus type.

In one particular embodiment, the direct colouring system comprises at least one natural colouring agent, especially as defined above, in particular a water-soluble colouring agent.

The composition may also comprise pigments and/or nacres in dispersed form, with the proviso that they do not affect the expected properties of the composition.

Advantageously, these insoluble colouring agents may be present in a proportion of less than 8% by weight, more particularly less than 5% and especially less than 3% by weight relative to the total weight of the composition.

Non-limiting illustrations of mineral pigments that may be mentioned more particularly include yellow, red or brown metal oxides, for instance iron oxides.

As metal powders, mention may be made of copper powder.

The pigments used in the context of the present invention may be used either in their crude form or in a pretreated form, especially treated at their surface.

Representative surface treatments that may especially be mentioned include that which consists in treating the pigment with a hydrophobic and oil-repellent agent of perfluoroalkyl phosphate type as described in patent EP 1 086 683.

As illustrations of pigments that are more particularly suitable for the invention, mention may be made especially of brown iron oxide and yellow iron oxide, coated with perfluoroalkyl phosphate, and alumina-treated titanium oxide, coated with perfluoroalkyl phosphate, in particular such as the pigmentary pastes sold under the trade names Yellow Iron Oxide Covafluor, PF5 Yellow 601 (yellow) and PF5 R516L (red) by the company Daito, and under the trade names FA50DRF, FA50DYF, FA65DF and FA65 DBF by the company Kobo.

The term “nacres” should be understood as meaning coloured particles of any form, which may or may not be iridescent, especially produced by certain molluscs in their shell, or alternatively synthesized, and which have a colour effect via optical interference.

The nacres may be chosen from nacreous pigments such as titanium mica coated with an iron oxide, mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

The nacres may more particularly have a yellow, pink, red, bronze, orange, brown and/or coppery colour or tint.

As illustrations of nacres that may be used in the context of the present invention, mention may be made in particular of gold-coloured nacres sold especially by the company Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold especially by the company Merck under the names Bronze fine (17384) (Colorona) and Bronze (17353) (Colorona) and by the company Engelhard under the name Super bronze (Cloisonne); the orange nacres sold especially by the company Engelhard under the names Orange 363C (Cloisonne) and Orange MCR 101 (Cosmica) and by the company Merck under the names Passion orange (Colorona) and Matte orange (17449) (Microna); the brown-tinted nacres sold especially by the company Engelhard under the names Nu-antique copper 340XB (Cloisonne) and Brown CL4509 (Chromalite); the nacres with a copper tint sold especially by the company Engelhard under the name Copper 340A (Timica); the nacres with a red tint sold especially by the company Merck under the name Sienna fine (17386) (Colorona); the nacres with a yellow tint sold especially by the company Engelhard under the name Yellow (4502) (Chromalite); the red-tinted nacres with a golden tint sold especially by the company Engelhard under the name Sunstone G012 (Gemtone); the pink nacres sold especially by the company Engelhard under the name Tan opale G005 (Gemtone); the black nacres with a golden tint sold especially by the company Engelhard under the name Nu antique bronze 240 AB (Timica); the blue nacres sold especially by the company Merck under the name Matte blue (17433) (Microna); the white nacres with a silvery tint sold especially by the company Merck under the name Xirona Silver; and the golden-green pinkish-orange nacres sold especially by the company Merck under the name Indian summer (Xirona), and mixtures thereof.

According to a specific embodiment, the composition according to the invention contain less than 2%, in particular less than 1%, more particularly less than 0.5% by weight relative to their total weight of reflective particles.

More particularly, they are free of reflective particles.

According to the invention, on reflective particles is a particle able to provide a highlight point that is visible to the make of eye.

The compositions according to the invention may also comprise as additional colouring agent one or more compounds that exert a self-tanning action on human skin, like derivatives containing a ketone group, for instance hydroxymethyl ketones and in particular dihydroxyacetone (DHA) and also methylglyoxane.

Physiologically Acceptable Medium

The term “physiologically acceptable medium” denotes a non-toxic medium that may be applied to human skin. The physiologically acceptable medium is generally suited to the nature of the skin onto which the composition is to be applied, and also to the form in which the composition is intended to be conditioned, especially in the form that is fluid at room temperature and atmospheric pressure.

As stated previously, the compositions of the invention are more particularly compositions intended for making up the skin and especially facial skin. They may thus be base compositions and/or compositions of foundation type.

They may be solid or liquid and are advantageously formulated in a fluid form. Thus, they may be in an anhydrous form or in the form of a gel or a direct, inverse or multiple emulsion combining at least one aqueous phase and at least one fatty phase.

As stated previously, they advantageously have a viscosity of greater than 100 centipoises and in particular greater than or equal to 300 centipoises.

This viscosity may be measured by the following method.

The measurement is performed using a Rheomat RM 180 type viscometer. The product is poured into a cup appropriate for the rotor used, according to the fluidity of the product. The viscosity is measured after 10 minutes of rotation, at a speed of 75 s⁻¹, and at room temperature.

Aqueous Phase

The compositions according to the invention may comprise at least one aqueous medium, constituting an aqueous phase, which may form the continuous phase of the composition under consideration.

The aqueous phase may consist essentially of water.

It may also comprise a mixture of water and of water-miscible organic solvent (miscibility in water of greater than 50% by weight at 25° C.), for instance lower monoalcohols containing from 1 to 5 carbon atoms, such as ethanol or isopropanol, glycols containing from 2 to 8 carbon atoms such as propylene glycol, ethylene glycol, 1,3-butylene glycol or dipropylene glycol, C₃-C₄ ketones and C₂-C₄ aldehydes.

According to one particular embodiment, the solvent of lower monoalcohol type in the context of the present invention may represent from 1% to 70% and preferentially from 2% to 50% by weight of the composition used in the claimed process.

As monoalcohols that are suitable for use in the invention, mention may be made of saturated hydrocarbon-based compounds comprising only one hydroxyl function and containing from 2 to 15 carbon atoms and in particular from 4 to 12 carbon atoms.

As examples of monoalcohols that are suitable for the invention, mention may be made, in a non-limiting manner, of ethanol, propanol, isopropanol, butanol, pentanol, propanol, hexanol, heptanol, octanol, decanol and dodecanol.

The term “polyhydroxylated solvent” is intended to denote any hydrocarbon-based compound comprising at least two hydroxyl functions and containing from 4 to 40 carbon atoms, in particular from 6 to 36 carbon atoms, in particular from 8 to 32 carbon atoms, in particular from 16 to 28 carbon atoms and more particularly from 18 to 24 carbon atoms.

The hydrocarbon-based chains may be interrupted, where appropriate, by the presence of at least one heteroatom, and especially an oxygen atom.

The polyol that is suitable for use in the present invention may be chosen especially from linear, branched, cyclic or polycyclic, saturated or unsaturated alcohols.

Thus, the polyol may be chosen, for example, from a diol, a triol, a tetraol, or a pentaol, or an ester thereof.

The polyol may be a diol, or an ester thereof, chosen especially from a fatty alcohol dimer, a monoglycerol or polyglycerol, a C₂₋₄ monoalkylene or polyalkylene glycol, 1,4-butanediol and pentaerythritol.

As examples of diols that are also suitable for use in the invention, mention may be made, in a non-exhaustive manner, of butanediol, pentanediol, propanediol, hexanediol, hexylene glycol, heptanediol, octanediol, nonanediol, decanediol, undecanediol, dodecanediol, tridecanediol, tetradecanediol, pentadecanediol, hexadecanediol, nonadecanediol, octadecanediol, cyclohexanediol, 1,1′-oxydipropanediol, diglycerol, erythritol, pentaerythritol, xylitol, sorbitol, phytanetriol (3,7,11,15-tetramethyl-1,2,3-trihydroxyhexadecane), ethylene glycol and xylene glycol, and isomers thereof.

A mixture that is most particularly suitable is a mixture based on at least ethanol and propylene glycol or dipropylene glycol or butylene glycol.

The aqueous phase (water and optionally the water-miscible organic solvent(s)) may be present in a content ranging from 1% to 99% by weight, especially ranging from 3% to 80% by weight, and in particular ranging from 5% to 60%, by weight relative to the total weight of the composition under consideration.

Fatty Phase

The composition according to the invention may comprise a fatty phase and especially at least one fatty substance that is liquid at room temperature (25° C.) and/or a fatty substance that is solid at room temperature, such as waxes, pasty fatty substances and gums, and mixtures thereof. The fatty phase may also contain lipophilic organic solvents.

The composition may contain, for example, a continuous fatty phase, which may contain less than 5% water and especially less than 1% water, relative to its total weight, and may in particular be in anhydrous form.

The fatty phase of the composition according to the invention may especially comprise, as liquid fatty substance, at least one volatile or non-volatile oil or a mixture thereof.

For the purposes of the invention, the term “volatile oil” means any oil capable of evaporating on contact with the skin in less than one hour, at room temperature and atmospheric pressure. The volatile oils of the invention are volatile cosmetic oils, which are liquid at room temperature, having a non-zero vapour pressure, at room temperature and atmospheric pressure, ranging in particular from 0.01 to 300 mmHg (1.33 Pa to 40 000 Pa) and preferably greater than 0.3 mmHg (30 Pa).

The term “non-volatile oil” means an oil that remains on the skin at room temperature and atmospheric pressure for at least several hours and that especially has a vapour pressure of less than 0.01 mmHg (1.33 Pa).

These volatile or non-volatile oils may be hydrocarbon-based oils, silicone oils or mixtures thereof. The term “hydrocarbon-based oil” means an oil mainly containing hydrogen and carbon atoms and possibly oxygen, nitrogen, sulfur and phosphorus atoms.

The volatile hydrocarbon-based oils may be chosen from hydrocarbon-based oils containing from 8 to 16 carbon atoms, and especially branched C₈-C₁₆ alkanes, for instance C₈-C₁₆ isoalkanes of petroleum origin (also known as isoparaffins), for instance isododecane (also known as 2,2,4,4,6-pentamethylheptane), isodecane, isohexadecane, and for example the oils sold under the trade names Isopars® or Permetyls®, branched C₈-C₁₆ esters such isohexyl neopentanoate, and mixtures thereof. Other volatile hydrocarbon-based oils, for instance petroleum distillates, especially those sold under the name Shell Solt® by the company Shell, may also be used.

Volatile oils that may also be used include volatile silicones, for instance volatile linear or cyclic silicone oils, especially those with a viscosity≦8 centistokes (8×10⁻⁶ m²/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyl-disiloxane, octamethyltrisiloxane, decamethyltetrasiloxane and dodecamethylpentasiloxane, and mixtures thereof.

The volatile oil may be present in a composition according to the invention in a content ranging from 0.1% to 98% by weight, especially from 1% to 65% by weight, and in particular from 2% to 50% by weight, relative to the total weight of the composition.

The non-volatile oils may be chosen especially from non-volatile fluoro and/or silicone hydrocarbon-based oils.

Non-volatile hydrocarbon-based oils that may especially be mentioned include:
hydrocarbon-based oils of animal origin,
hydrocarbon-based oils of plant origin, such as triglycerides consisting of fatty acid esters of glycerol, the fatty acids of which may have varied chain lengths from C₄ to C₂₄, these chains possibly being linear or branched, and saturated or unsaturated; these oils are especially wheatgerm oil, sunflower oil, grapeseed oil, sesameseed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, sesame oil, marrow oil, rapeseed oil, blackcurrant oil, evening primrose oil, millet oil, barley oil, quinoa oil, rye oil, safflower oil, candlenut oil, passionflower oil or musk rose oil; shea butter; or caprylic/capric acid triglycerides, for instance those sold by the company Stéarineries Dubois or those sold under the names Miglyol 810, 812 and 818® by the company Dynamit Nobel,
synthetic ethers containing from 10 to 40 carbon atoms,
linear or branched hydrocarbons of mineral or synthetic origin, such as petroleum jelly, polydecenes, hydrogenated polyisobutene such as Parleam, and squalane, and mixtures thereof,
synthetic esters, for instance oils of formula R₁COOR₂ in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂≧10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, alcohol or polyalcohol heptanoates, octanoates, decanoates or ricinoleates, for instance propylene glycol dioctanoate; hydroxylated esters, for instance isostearyl lactate or diisostearyl malate; polyol esters and pentaerythritol esters,
fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol,
higher fatty acids such as oleic acid, linoleic acid or linolenic acid, and mixtures thereof.

The non-volatile silicone oils that may be used in the composition according to the invention may be non-volatile polydimethylsiloxanes (PDMS), polydimethylsiloxanes comprising alkyl or alkoxy groups, which are pendent and/or at the end of a silicone chain, these groups each containing from 2 to 24 carbon atoms, phenylsilicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates.

The non-volatile oils may be present in a composition according to the invention in a content ranging from 0.01% to 90% by weight, especially from 0.1% to 85% by weight and in particular from 1% to 70% by weight relative to the total weight of the composition.

More generally, the liquid fatty substance may be present in a proportion of from 0.01% to 90% by weight and especially from 0.1% to 85% by weight relative to the weight of the fatty phase.

As regards the fatty substance that is solid at room temperature and atmospheric pressure, it may be chosen from waxes, pasty fatty substances and gums, and mixtures thereof. This solid fatty substance may be present in a proportion of from 0.01% to 50%, especially from 0.1% to 40% and in particular from 0.2% to 30% by weight relative to the total weight of the fatty phase.

Thus, a composition according to the invention may comprise at least one fatty substance that is pasty at room temperature.

For the purposes of the invention, the term “pasty fatty substance” means fatty substances with a melting point ranging from 20 to 55° C. and preferably 25 to 45° C., and/or a viscosity at 40° C. ranging from 0.1 to 40 Pa.s (1 to 400 poises) and preferably 0.5 to 25 Pa.s, measured using a Contraves TV or Rheomat 80 viscometer, equipped with a spindle rotating at 60 Hz. A person skilled in the art can select the spindle for measuring the viscosity from the spindles MS-r3 and MS-r4, on the basis of his general knowledge, so as to be able to perform the measurement on the pasty compound tested.

Preferably, these fatty substances are hydrocarbon-based compounds optionally of polymeric type; they may also be chosen from silicone compounds; they may also be in the form of a mixture of hydrocarbon-based and/or silicone compounds. In the case of a mixture of different pasty fatty substances, the hydrocarbon-based pasty compounds (mainly containing carbon and hydrogen atoms and optionally ester groups) are preferably used, in majority proportion.

Among the pasty compounds that may be used in the composition according to the invention, mention may be made of lanolins and lanolin derivatives, for instance acetylated lanolins, oxypropylenated lanolins or isopropyl lanolate, with a viscosity of from 18 to 21 Pa.s and preferably 19 to 20.5 Pa.s, and/or a melting point of from 30 to 55° C., and mixtures thereof. Esters of fatty acids or of fatty alcohols, especially those containing 20 to 65 carbon atoms (melting point from about 20 to 35° C. and/or viscosity at 40° C. ranging from 0.1 to 40 Pa.s) may also be used, for instance triisostearyl citrate or cetyl citrate; arachidyl propionate; polyvinyl laurate; cholesterol esters, for instance triglycerides of plant origin such as hydrogenated plant oils, viscous polyesters, for instance poly(12-hydroxystearic acid), and mixtures thereof. Triglycerides of plant origin that may be used include hydrogenated castor oil derivatives, such as “Thixinr” from Rheox.

Mention may also be made of silicone pasty fatty substances such as polydimethylsiloxanes (PDMS) of high molecular weight and in particular those with pendent chains of the alkyl or alkoxy type containing from 8 to 24 carbon atoms, and a melting point of 20-55° C., for instance stearyl dimethicones, especially those sold by the company Dow Corning under the trade names DC2503® and DC25514®, and mixtures thereof.

The pasty fatty substance may be present in a composition according to the invention in a content ranging from 0.01% to 50% by weight, preferably ranging from 0.1% to 45% by weight and better still ranging from 0.2% to 30% by weight relative to the total weight of the said composition.

The composition according to the invention may also comprise a wax. The wax may be solid at room temperature (25° C.), with a reversible solid/liquid change of state, having a melting point of greater than 30° C. which may be up to 200° C., a hardness of greater than 0.5 MPa and having an anisotropic crystal organization in the solid state. It may be a hydrocarbon-based wax, a fluoro wax and/or a silicone wax and may be of animal, plant, mineral or synthetic origin. It may be chosen, for example, from beeswax, carnauba wax, candelilla wax, paraffin waxes, hydrogenated castor oil, silicone waxes or microcrystalline waxes, and mixtures thereof.

In particular, the wax may be present in the form of a wax-in-water emulsion.

The wax may be present in a composition according to the invention in a content ranging from 0.01% to 50% by weight, in particular from 0.1% to 30% by weight and especially from 0.2% to 20% by weight relative to the total weight of the composition.

Surfactants

The composition may also contain emulsifying surfactants present especially in a proportion ranging from 0.1% to 30% by weight and better still from 5% to 15% by weight relative to the total weight of the composition.

These surfactants may be chosen from anionic and nonionic surfactants. Reference may be made to the document “Encyclopedia of Chemical Technology, Kirk-Othmer”, volume 22, pp. 333-432, 3rd edition, 1979, Wiley, for the definition of the properties and functions (emulsifying) of surfactants, in particular pp. 347-377 of this reference, for the anionic and nonionic surfactants.

The surfactants preferably used in the composition of foundation type according to the invention are chosen from:
nonionic surfactants: fatty acids, fatty alcohols, polyethoxylated or polyglycerolated fatty alcohols such as polyethoxylated stearyl or cetylstearyl alcohol, fatty acid esters of sucrose, alkylglucose esters, in particular polyoxyethylenated C₁-C₆ alkyl glucose fatty esters, and mixtures thereof,
anionic surfactants: C₁₆-C₃₀ fatty acids neutralized with amines, aqueous ammonia or alkaline salts, and mixtures thereof,
silicone surfactants chosen from the compounds of general formula (I) below:

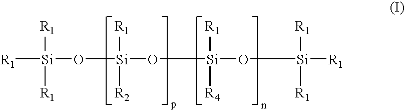

in which formula:
R₁ and R₄, which may be identical or different, represent a hydrogen atom, a linear or branched C₁-C₃₀ alkyl radical or a phenyl radical,
R₂, which may be identical or different, represent —(CxH₂x)-(OC₂H₄)a-(OC₃H₆)b-OR₃,
R₃, which may be identical or different, are chosen from a hydrogen atom, a linear or branched alkyl radical containing from 1 to 12 carbon atoms, and a linear or branched acyl radical containing from 2 to 12 carbon atoms,
n ranges from 1 to 1000,
p ranges from 1 to 30,
a ranges from 1 to 50,
b ranges from 1 to 50,
x ranges from 1 to 5.

The number-average molecular weight of this silicone emulsifier is generally greater than or equal to 15 000 and preferably between 20 000 and 40 000.

A first family of polyalkyl polyether siloxanes that is particularly suited to the compositions according to the invention is that of the compounds corresponding to formula (I) above for which the radicals R₁ and R₄ are identical and both represent methyl radicals and the radical R₃ represents hydrogen.

As examples of silicone emulsifiers belonging to this family, mention may be made of oxyethylenated oxypropylenated (18/18 EO/PO) polydimethyl/methylsiloxane for which n is 396 and p is 4, with a number-average molecular weight of greater than 30 000 (CTFA name: 90% cyclomethicone/10% dimethicone copolyol) sold under the trade name Silicone Q2-3225C by the company Dow Corning.

In the above definition and in the text hereinbelow, EO represents one mole of ethylene oxide and PO represents one mole of propylene oxide.

A second family of polyalkyl polyether siloxanes that is particularly suited to the compositions according to the invention is that of the compounds corresponding to formula (I) above for which the radicals R₁ all represent methyl radicals and the radicals R₄ all represent lauryl radicals.

A particularly preferred silicone emulsifier of this second family is oxyethylenated oxypropylenated (18/18 EO/PO) polymethyllauryl/methylsiloxane for which n is 35 and p is 3, with a number-average molecular weight of greater than 25 000 (CTFA name: 91% lauryl methicone copolyol/9% isostearyl alcohol) sold under the trade name DC Q2-5200 by the company Dow Corning.

A silicone emulsifier that is most particularly preferred for the compositions according to the invention is an α,ω-substituted oxyalkylene silicone of linear structure, substituted at the two ends of the main chain with oxyalkylene groups linked to the Si atoms via a hydrocarbon-based group. Mention will be made more particularly of a silicone corresponding to the general formula (II) below:

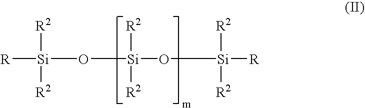

in which:

R=—(CH₂)s-O—(C₂H₄O)t-(C₃H₆O)uR₁

in which:
R₁ represents H, CH₃ or CH₂CH₃,
s is an integer ranging from 1 to 5, t ranges from 1 to 100 and u ranges from 0 to 50,
the units (C₂H₄O) and (C₃H₆O) possibly being distributed randomly or in blocks,

or
the radicals R² represent a C₁-C₃ alkyl radical or a phenyl radical,
5=m=300.

Preferably, the α,ω-substituted oxyalkylenated silicone used according to the present invention corresponds to the general formula (II) for which all the radicals R² are methyl radicals and:
s ranges from 2 to 4,
t ranges from 3 to 100,
m ranges from 50 to 200.

More preferably, the average molecular weight of R ranges from 800 to 2600.

Preferably, the weight ratio of the C₂H₄O units relative to the C₃H₆O units ranges from 100:10 to 20:80. Advantageously, this ratio is about 42/58.

More preferably, R₁ is a methyl group.

Even more preferentially, the emulsion according to the invention comprises the α,ω-oxyalkylenated silicone having the following formula:

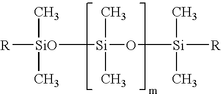

in which:
m=100,
R═(CH₂)₃—O—(C₂H₄O)x-(C₃H₆O)y-CH₃, in which t ranges from 3 to 100, u ranges from 1 to 50, the weight ratio of the number of C₂H₄O to the number of C₃H₆O being about 42/58, the average molecular weight of R ranging from 800 to 1000.

Among the commercial products that may contain all or the part of the α,ω-substituted oxyalkylenated silicones that may be used according to the invention as emulsifier, mention may be made especially of those sold under the names Abil EM 97 by the company Goldschmidt or KF 6009, X22-4350, X22-4349 or KF 6008 by the company Shin-Etsu.

The polyalkyl polyether siloxane emulsifier as defined above is used according to the invention in a preferential proportion ranging from 0.1% to 30% and more particularly from 0.5% to 10% by weight relative to the total weight of the emulsion.

Film-Forming Polymer

The composition according to the invention may also comprise at least one film-forming polymer.

According to the present invention, the term “film-forming polymer” means a polymer capable of forming, by itself or in the presence of an auxiliary film-forming agent, a continuous film that adheres to the skin.

A film-forming polymer capable of forming a hydrophobic film, i.e. a polymer whose film has a solubility in water at 25° C. of less than 1% by weight, is preferably used.

The film-forming polymer may especially be at least one polymer chosen from the group comprising:
water-soluble film-forming polymers,
aqueous dispersions of water-dispersible film-forming polymer particles, also known as “latices”; in this case, the composition should comprise an aqueous phase,
liposoluble film-forming polymers,
lipodispersible film-forming polymers in the form of non-aqueous dispersions of polymer particles, preferably dispersions of polymer particles, where appropriate surface-stabilized with at least one stabilizer, in one or more silicone and/or hydrocarbon-based oils; these non-aqueous dispersions are also known as “NADs”.

The film-forming polymer may be present in a composition according to the invention in a solids content ranging from 0.01% to 20% by weight and especially from 0.5% to 10% by weight relative to the total weight of the composition.

Among the film-forming polymers that may be used according to the invention, mention may be made of synthetic polymers, of free-radical type or of polycondensate type, polymers of natural origin, and mixtures thereof.

The term “free-radical film-forming polymer” means a polymer obtained by polymerization of unsaturated monomers, especially ethylenically unsaturated monomers, each monomer being capable of homopolymerizing (unlike polycondensates). The film-forming polymers of free-radical type may especially be vinyl polymers or copolymers, especially acrylic polymers.

Aqueous dispersions of film-forming polymers that may be used include the acrylic dispersions sold under the names Neocryl XK-90®, Neocryl A-1070®, Neocryl A-1090®, Neocryl BT-62®, Neocryl A-1079®, Neocryl A-523® by the company Avecia-Neoresins, Dow Latex 432® by the company Dow Chemical, Daitosol 5000 AD® by the company Daito Kasey Kogyo; or alternatively the aqueous polyurethane dispersions sold under the names Neorez R-981®, Neorez R-974® by the company Avecia-Neoresins, Avalure UR-405®, Avalure UR-410®, Avalure UR-425®, Avalure UR-450®, Sancure 875®, Sancure 861®, Sancure 878®, Sancure 2060® by the company Goodrich, Impranil 85® by the company Bayer and Aquamere H-1511® by the company Hydromer.

Examples of water-soluble film-forming polymers that may be mentioned include proteins, anionic, cationic, amphoteric or nonionic chitin or chitosan polymers, cellulose polymers, acrylic polymers or copolymers, vinyl polymers, and polymers of natural origin, which are optionally modified, and mixtures thereof.

The film-forming polymer may also be present in the form of surface-stabilized particles, dispersed in the liquid fatty phase. Dispersions of film-forming polymer in the liquid fatty phase, in the presence of stabilizers, are especially described in documents EP-A-749 746, EP-A-923 928 and EP-A-930 060.

As liposoluble film-forming polymers that may be used in the invention, mention may also be made of polyalkylenes and especially copolymers of C₂-C₂₀ alkenes, for instance polybutene, alkylcelluloses with a saturated or unsaturated, linear or branched C₁ to C₈ alkyl radical, for instance ethylcellulose and propylcellulose, vinylpyrrolidone (VP) copolymers and especially copolymers of vinylpyrrolidone and of a C₂ to C₄₀ and better still C₃ to C₂₀ alkene. As examples of VP copolymers that may be used in the invention, mention may be made of the VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate copolymer.

The composition according to the invention may also comprise an auxiliary film-forming agent that promotes the formation of a film with the film-forming polymer. Such a film-forming agent may be chosen from any compound known to the those skilled in the art as being capable of satisfying the desired function, and may be chosen especially from plasticizers and coalescers.

Filler

Needless to say, it nevertheless remains possible to introduce into the composition according to the invention one or more conventional fillers, with the proviso that these conventional fillers are used in an amount such that they do not affect the aesthetic effect desired by the compositions under consideration according to the invention, i.e. they do not excessively impart a grayish appearance to the made-up skin when this skin is coated with a composition in accordance with the invention. A person skilled in the art is capable, by means of his knowledge, of making this adjustment.

These fillers may be mineral or organic and of any form: platelet, spherical or oblong, irrespective of the crystallographic form (for example leaflet, cubic, hexagonal, orthorhombic, etc.). Mention may be made of talc, mica, silica, kaolin, polyamide powder (Nylon®) (Orgasol® from Atochem), poly-β-alanine powder and polyethylene powder, tetrafluoroethylene polymer powders (Teflon®), lauroyllysine, starch, boron nitride, hollow polymer microspheres such as those made of polyvinylidene chloride/acrylonitrile, for instance Expancel® (Nobel Industrie), made of acrylic acid copolymers (Polytrap® from the company Dow Corning) or made of polymethyl methacrylate (Covabead from Wackherr), silicone resin microbeads (for example Tospearls® from Toshiba), polyorganosiloxane elastomer particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc, magnesium or lithium stearate, zinc laurate or magnesium myristate, and mixtures thereof.

In the present case, the compositions in accordance with the invention may contain less than 15% or even less than 5% by weight of filler, or may even be free of filler.

It may be advantageous to favour the choice of transparent fillers, for instance fumed silica or polymethyl methacrylate (PMMA).

The composition under consideration according to the invention is generally in the form of a makeup base composition, a foundation especially to be applied to the face or the neck, a concealer product, a complexion corrector, a tinted cream or facial makeup base, or a body makeup composition.

The composition according to the invention may be in the form of a fluid, for example pasty or liquid. It may be in the form of a soft paste, an ointment or a solid or fluid pomade of cream type. For example, it may be an oil-in-water, water-in-oil or multiple emulsion, a solid emulsion especially of water-in-oil type, a gel, especially a solid or soft anhydrous gel, and even in two-phase form.

It may also be in a solid form, for example pulverulent, compacted or cast, or in stick form.

The invention may be understood more clearly on reading the detailed description that follows, of non-limiting implementation examples thereof.

### EXAMPLES

### Example 1

### Cosmetic Composition of Coloured Oil-in-Water Emulsion Type

| | | |
|---|---|---|
| A | Mixture of (linear C14-15) dialkyl tartrate, cetylstearyl alcohol and | 1.500 |
| | oxyethylenated (25 EO) oxypropylenated (2) lauryl alcohol | |
| | (Cosmacol PSE from Sasol) | |
| | Mixture of glyceryl mono/distearate/polyethylene glycol stearate | 2.000 |
| | (100 EO) (Arlacel 165 FL from Uniqema) | |
| | Stearyl alcohol (95% C18) (Lanette 18 from Cognis) | 1.000 |
| | Cyclohexadimethylsiloxane (Dow Corning 246 Fluid from Dow Corning) | 7.500 |
| | Hydrogenated isoparaffin (6-8 mol of isobutylene) (Parleam from | 2.500 |
| | NOF Corporation) | |
| | Butyl p-hydroxybenzoate (distributed by Ueno Fine Chemicals) | 0.150 |
| | Protected 2-ethylhexyl 4-methoxycinnamate (Parsol MCX from | 1.000 |
| | DSM Nutritional Products) | |
| B1 | Water | 52.500 |
| | Methyl p-hydroxybenzoate (distributed by NIPA) | 0.250 |
| | Chlorphenesin (from Denk (Merck)) | 0.250 |
| B2 | Water | 16.350 |
| | Disodium salt of tartrazine (CI: 19140) (08005 FD & C Yellow 5 | 0.255 |
| | from LCW (Sensient) | |
| | Disodium salt of brilliant yellow FCF (CI: 15985) (37001 FD & C | 0.302 |
| | Yellow No. 6 from Univar) | |
| | Disodium salt of brilliant green FCF (CI: 42053) (06503 FD & C | 0.035 |
| | Green 3 from LCW (Sensient) | |
| | Disodium salt of fuchsin acid D (CI: 17200) (K7057 D & C Red 33 | 0.158 |
| | from LCW Sensient) | |
| C | Water | 1.000 |
| | Chlorhexidine digluconate in solution (chlorhexidine digluconate | 0.250 |
| | 20% solution BP/PH. EUR. from Schutz Dishman Biotech) | |
| D | Water | 9.750 |
| | Sodium hyaluronate (MW: 1 100 000) in powder form | 0.250 |
| | (Crystalhyal from Soliance) | |
| E | Corn starch esterified with octenylsuccinic anhydride, aluminium | 3.000 |
| | salt (Dry-Flo Plus (28-1160) (Europe) from National Starch) | |

Procedure:

Phase A is weighed out in a 400 mL beaker, which is then placed on a water bath at 75° C. until fully dissolved.

The various constituents of phase B1 are weighed out in the main beaker (800 mL) and phase B2 is weighed out in a 250 mL beaker, and they are then placed on a water bath at 75° C. until fully dissolved. When the two phases have correctly dissolved, B2 is added to B1 with magnetic stirring.

Phase D (gel) is prepared separately using a Rayneri blender.

The emulsion forms at 75° C. To do this, the fatty phase A is poured into the aqueous phase B, while gradually increasing the stirring speed to 1500 rpm. There should always be a small vortex. Mechanical stirring is continued for 10 minutes.

The temperature is then reduced to 45° C. and phase C is added (stirring for 5 minutes), followed by phase D (stirring for 5 minutes) and then phase E (stirring for 10 minutes).

This colouring base allows a neutral brown shade to be obtained on dark skin. The covering power (Contrast Ratio) measured with this colouring base is 24.23 and its viscosity is 346 centipoises.

### Example 2

### Cosmetic Composition of Coloured Oil-in-Water Emulsion Type

| | | |
|---|---|---|
| A | Mixture of (linear C14-15) dialkyl tartrate, cetylstearyl alcohol and | 1.500 |
| | oxyethylenated (25 EO) oxypropylenated (2) lauryl alcohol (Cosmacol PSE | |
| | from Sasol) | |
| | Mixture of glyceryl mono/distearate/polyethylene glycol stearate (100 | 2.000 |
| | EO) (Arlacel 165 FL from Uniqema) | |
| | Stearyl alcohol (95% C18) (Lanette 18 from Cognis) | 1.000 |
| | Cyclohexadimethylsiloxane (Dow Corning 246 Fluid from Dow | 7.500 |
| | Corning) | |
| | Hydrogenated isoparaffin (6-8 mol of isobutylene) (Parleam from NOF | 2.500 |
| | Corporation) | |
| | Butyl p-hydroxybenzoate (distributed by Ueno Fine Chemicals) | 0.150 |
| | Protected 2-ethylhexyl 4-methoxycinnamate (Parsol MCX from DSM | 1.000 |
| | Nutritional Products) | |
| B1 | Water | 52.500 |
| | Methyl p-hydroxybenzoate (distributed by NIPA) | 0.250 |
| | Chlorphenesin (from Denk (Merck)) | 0.250 |
| B2 | Water | 16.129 |
| | Disodium salt of ponceau SX (CI: 14700) (07004 FD & C Red 4 from | 0.385 |
| | LCW (Sensient)) | |
| | Disodium salt of brilliant yellow FCF (CI: 15985) (37001 FD & C | 0.500 |
| | Yellow No. 6 from Univar) | |
| | Disodium salt of brilliant green FCF (CI: 42053) (06503 FD & C | 0.048 |
| | Green 3 from LCW (Sensient) | |
| | Disodium salt of fuchsin acid D (CI: 17200) (K7057 D & C Red 33 | 0.038 |
| | from LCW Sensient) | |
| C | Water | 1.000 |
| | Chlorhexidine digluconate in solution (chlorhexidine digluconate 20% | 0.250 |
| | solution BP/PH. EUR. from Schutz Dishman Biotech) | |
| D | Water | 9.750 |
| | Sodium hyaluronate (MW: 1 100 000) in powder form (Crystalhyal | 0.250 |
| | from Soliance) | |
| B | Corn starch esterified with octenylsuccinic anhydride, aluminium salt | 3.000 |
| | (Dry-Flo Plus (28-1160) (Europe) from National Starch) | |

The procedure is the same as that of Example 1.

This colouring base allows a red-brown shade to be obtained on dark skin. The covering power (Contrast Ratio) measured with this colouring base is 25.5 and its viscosity is 490 centipoises.

### Example 3

### Cosmetic Composition of Coloured Oil-in-Water Emulsion Type Based on Natural Dyes

| | | |
|---|---|---|
| A | Mixture of (linear C14-15) dialkyl tartrate, cetylstearyl alcohol and | 1.500 |
| | oxyethylenated (25 EO) oxypropylenated (2) lauryl alcohol (Cosmacol PSE | |
| | from Sasol) | |
| | Mixture of glyceryl mono/distearate/polyethylene glycol stearate (100 | 2.000 |
| | EO) (Arlacel 165 FL from Uniqema) | |
| | Stearyl alcohol (95% C18) (Lanette 18 from Cognis) | 1.000 |
| | Cyclohexadimethylsiloxane (Dow Corning 246 Fluid from Dow | 7.500 |
| | Corning) | |
| | Hydrogenated isoparaffin (6-8 mol of isobutylene) (Parleam from NOF | 2.500 |
| | Corporation) | |
| | Butyl p-hydroxybenzoate (distributed by Ueno Fine Chemicals) | 0.150 |
| | Protected 2-ethylhexyl 4-methoxycinnamate (Parsol MCX from DSM | 1.000 |
| | Nutritional Products) | |
| B1 | Water | 50.000 |
| | Methyl p-hydroxybenzoate (distributed by NIPA) | 0.250 |
| | Chlorphenesin (from Denk (Merck)) | 0.250 |
| B2 | Water | 3.615 |
| | Beetroot extract (Beetroot juice P-WS (4/96) from LCW (Sensient) | 13.760 |
| | Beta-carotene extract (B-Carotene 10% P-WS) from LCW (Sensient) | 2.190 |
| | Sodium salt of cupric chlorophylline in powder form (97/3) from LCW | 0.035 |
| | (Sensient) | |
| C | Water | 1.000 |
| | Chlorhexidine digluconate in solution (chlorhexidine digluconate 20% | 0.250 |
| | solution BP/PH. EUR. from Schutz Dishman Biotech) | |
| D | Water | 9.750 |
| | Sodium hyaluronate (MW: 1 100 000) in powder form (Crystalhyal | 0.250 |
| | from Soliance) | |
| E | Corn starch esterified with octenylsuccinic anhydride, aluminium salt | 3.000 |
| | (Dry-Flo Plus (28-1160) (Europe) from National Starch) | |
| | The procedure is the same as that of Example 1. | |
| | This colouring base allows a bronze shade to be obtained on dark skin. | |

### Example 4

### Composition of Foundation Type in the Form of a Solid Emulsion

| | | |
|---|---|---|
| A1 | Purified candelilla wax (NC 1630 from Cera Rica Noda) | 1.800 |
| | Hydrocarbon mineral wax (C20/C60) (Ozokerite wax SP 1020 P from | 1.200 |
| | Strahl & Pitsch) | |
| | Propyl p-hydroxybenzoate (from Nipa (Clariant)) | 0.300 |
| | Oxyethylenated poly methylcetyl dimethyl methylsiloxane (20/75/5 - | 2.000 |
| | viscosity: 3000 cSt) (Abil EM 90 from Goldschmidt (Degussa)) | |
| | Isotridecyl isononanoate (Crodamol TN (J) (ES02976) from Croda)) | 9.000 |
| | Polyglyceryl isostearate (4 mol) (Isolan GI 34 from Goldschmidt- | 1.500 |
| | Degussa) | |
| A2 | Ethers of dodecanediol (2.2 mol) and of polyethylene glycol (45 EO) | 4.000 |
| | (Elfacos ST 9 from Akzo Nobel) | |
| A3 | Cyclopentadimethylsiloxane (Dow Corning 245 Fluid from Dow | 28.625 |
| | Corning) | |
| | Mixture of oxyethylene oxypropylene (18 EO/18 PO) polydimethylsiloxane, | 2.000 |
| | cyclopentadimethylsiloxane and water (10/88/2) (Dow | |
| | Corning 5225C Formulation Aid from Dow Corning) | |
| A4 | Silica microbeads (SB150 from Miyoshi Kasei) | 9.000 |
| | Fumed silica of hydrophobic nature (Aerosil R 972 from LCW | 0.500 |
| | (Sensient)) | |
| | Hollow polymethyl methacrylate microspheres (particle size: 10 to 12 | 3.000 |
| | microns) (Covabead LH 85 from LCW (Sensient)) | |
| B1 | Water | 23.125 |
| | Methyl p-hydroxybenzoate (from Nipa) | 0.400 |
| | Chlorphenesin (from Denk (Merck)) | 0.300 |
| | 2-Phenoxyethanol (from SEPPIC) | 0.500 |
| | Magnesium sulfate heptahydrate (from Merck) | 1.000 |
| | Glycerol (Croderol GV 9000) (from Croda) | 5.000 |
| | 1,3-Butylene glycol | 3.000 |
| B2 | Water | 3.000 |
| | Disodium salt of tartrazine (CI: 19140) (08005 FD & C Yellow 5 from | 0.255 |
| | LCW (Sensient)) | |
| | Disodium salt of brilliant yellow FCF (CI: 15985) (37001 FD & C | 0.302 |
| | Yellow No. 6 from Univar) | |
| | Disodium salt of brilliant green FCF (CI: 42053) (06503 FD & C | 0.035 |
| | Green 3 from LCW (Sensient)) | |
| | Disodium salt of fuchsin acid D (CI: 17200) (K7057 D & C Red 33 | 0.158 |
| | from LCW Sensient) | |

Procedure:

Phase A1 is weighed out in a heating pan and melted until the mixture is clear. This mixture is stirred with a turbomixer and the temperature of the oil bath is lowered to about 80° C.

Phase A2 is added.

The pigments of phase A3 are moistened with the oils and then ground by milling three times in a three-roll mill. The ground material is incorporated with stirring into the mixture (A1+A2) that has been slightly cooled (temperature below 80° C.). The pigmentary paste is left stirring for about 30 minutes.

When the mixture (A1+A2+A3) has stabilized at a temperature of 68° C., phases A4 and then A5 are introduced. Mechanical homogenization is continued for 10 minutes.

In parallel, the aqueous phase B is prepared in a 400 ml of beaker, at a temperature of 68° C.

The emulsion is prepared at 67-68° C. with stirring by slowly pouring the aqueous phase into the fatty phase. Stirring is continued for 10 minutes.

The emulsion is then cast at 68° C., and then left in an oven at 20° C. for 24 hours.

This product allows a neutral brown shade to be obtained on dark skin.

1. A method for making up dark skin, comprising the application to the said skin of a composition comprising, in a physiologically acceptable medium, at least one direct colouring system in dissolved form, the said composition also having a viscosity of greater than 100 centipoises and a covering power, defined by the Contrast Ratio, of less than 80.

2. A method according to claim 1, wherein the composition has a viscosity of greater than 300 centipoises.

3. A method according to claim 1, wherein the said composition has a covering power, defined by the Contrast Ratio less than or equal to 50.

4. A method according to claim 1, wherein the direct colouring system is formed from (a) water-soluble or liposoluble colouring agent(s) or a mixture thereof.

5. A method according to claim 1, wherein the said direct colouring system comprises at least one dye selected from the group consisting of: DC Red 17, DC Red 21, DC Red 27, DC Green 6, DC Yellow 11, DC Violet 2, DC Orange 5, Sudan red, carotenes, xanthophylls, palm oil, Sudan brown, quinoline yellow, annatto, curcumin, FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin, carmine, cupric chlorophylline, methylene blue, anthocyanins, riboflavin, juglone, lawsone, caramel, extracts of fermented soya, of algae, of fungi or of microorganisms, flavylium salts not substituted in position 3, extracts of Gesneria fulgens, Blechum procerum or Saxifraga, and pigments that can be obtained by extraction with an organic or aqueous-organic solvent of a culture medium of micromycetes of the Monascus type.

6. A method according to claim 1, wherein the said direct colouring system comprises at least one natural colouring agent.

7. A method according to claim 1, wherein the composition comprises from 0.001% to 5% by weight of colouring agent(s) forming the direct colouring system.

8. A method according to claim 1, wherein the said composition affords a negative variation in lightness ΔL ranging from −20 to −1 and a variation in saturation ΔC* ranging from 0.5 to 20.

9. A method according to claim 1, wherein the said composition comprises at least one fatty phase.

10. A method according to claim 1, wherein the said composition comprises at least one aqueous phase.

11. A method according to claim 1, wherein the said composition is anhydrous.

12. A method according to claim 1, wherein the composition comprises at least one fatty substance that is liquid at room temperature and at atmospheric pressure and/or at least one fatty substance that is solid at room temperature and at atmospheric pressure.

13. A method according to claim 1, wherein the composition comprises at least one fatty substance that is liquid at room temperature and at atmospheric pressure comprises at least one volatile or non-volatile oil or a mixture thereof.

14. A method according to claim 1, wherein the composition comprises at least one fatty substance that is solid at room temperature and atmospheric pressure is chosen from waxes, pasty fatty substances and gums, and mixtures thereof.

15. A method according to claim 1, wherein the said composition comprises less than 15% by weight of filler(s).

16. A method according to claim 1, wherein the said composition also comprises at least pigments and/or nacres in dispersed form.

17. A method according to claim 1, wherein the said composition is in a fluid form of liquid, paste, direct or inverse emulsion, or gel type.

18. A method according to claim 1, wherein the said emulsion is in a solid form, or in stick form.

19. A method according to claim 1, wherein the said composition has a pH of greater than 5.

20. A method according to claim 1, wherein the said composition is in the form of a foundation to be applied to the face or the neck, a concealer product, a complexion corrector, a tinted cream or facial makeup base, or a body makeup composition.

21. A method for making up dark skin, comprising the application to the said skin of a composition comprising, in a physiologically acceptable medium, at least one direct colouring system in dissolved form, the said composition having a covering power, defined by the Contrast Ratio, of less than 80 and a pH of greater than 5.

22. A method according to claim 1, wherein the emulsion is compact, pulverulent or cast.
